(19) United States
(12) Patent Application Publication
(10) Pub. No.: US 2013/0324850 A1
(43) Pub. Date: Dec. 5, 2013

(54) SYSTEMS AND METHODS FOR INTERFACING WITH AN ULTRASOUND SYSTEM

(71) Applicants: Joe Petruzzelli, Paramus, NJ (US); John Judy, Marblehead, MA (US); Peter Schon, Paramus, NJ (US)

(72) Inventors: Joe Petruzzelli, Paramus, NJ (US); John Judy, Marblehead, MA (US); Peter Schon, Paramus, NJ (US)

(73) Assignee: MINDRAY DS USA, INC., Mahwah, NJ (US)

(21) Appl. No.: 13/485,238

(22) Filed: May 31, 2012

(51) Int. Cl. A61B 6/00; A61B 8/13

(52) U.S. Cl. 600/443; 600/407

(57) ABSTRACT

Systems and methods for enabling a user to interact with a medical imaging system using a touch screen display are disclosed. In certain embodiments, a touch screen display associated with the medical imaging system may receive input from a user based on a position of a contact point of the user with the touch screen display. The contact point may be located within a primary imaging area displaying images captured by the medical imaging system on the touch screen display. Based on the received input, a cursor may be displayed on the touch screen display within the primary imaging area in a particular position relative from the position of the contact point that is different than the position of the contact point.

### TECHNICAL FIELD

This disclosure relates to systems and methods for interfacing with a medical imaging system. Specifically, this disclosure relates to systems and methods for interfacing with an ultrasound imaging system that utilizes a touch screen interface.

### SUMMARY

Systems and methods are presented for enabling a user to interact with a medical imaging system using a touch screen display. In certain embodiments, a touch screen display associated with the medical imaging system may receive input from a user based on a position of a contact point of the user with the touch screen display. The contact point may be located within a primary imaging area displaying images captured by the medical imaging system on the touch screen display. Based on the received input, a cursor may be displayed on the touch screen display within the primary imaging area in a particular position relative from the position of the contact point that is different than the position of the contact point (e.g., in an offset position). By displaying the cursor in a position different than the contact point, a user may precisely position the cursor within the primary imaging area without obscuring a displayed area of interest.

### BRIEF DESCRIPTION OF THE DRAWINGS

FIG. 1 illustrates an exemplary interface for an ultrasound imaging system consistent with embodiments disclosed herein.

FIG. 2 illustrates an exemplary interface for an ultrasound imaging system including a cursor consistent with embodiments disclosed herein.

FIG. 3 illustrates an exemplary interface for an ultrasound imaging system including an off-set cursor consistent with embodiments disclosed herein.

FIG. 4 illustrates another exemplary interface for an ultrasound imaging system including an off-set cursor consistent with embodiments disclosed herein.

FIG. 5 illustrates an exemplary interface for an ultrasound imaging system including an annotation consistent with embodiments disclosed herein.

FIG. 6 illustrates an exemplary interface for an ultrasound imaging system including a rotatable cursor consistent with embodiments disclosed herein.

FIG. 7 illustrates an exemplary interface for an ultrasound imaging system including a user-defined region of interest consistent with embodiments disclosed herein.

FIG. 8 illustrates an exemplary interface for an ultrasound imaging system including a measurement system consistent with embodiments disclosed herein.

FIG. 9 illustrates an exemplary interface for an ultrasound imaging system including multi-segment tracing consistent with embodiments disclosed herein.

FIG. 10 illustrates another exemplary interface for an ultrasound imaging system including an annotation consistent with embodiments disclosed herein.

FIG. 11 illustrates another exemplary interface for an ultrasound imaging system including a cursor consistent with embodiments disclosed herein.

FIG. 12 illustrates another exemplary interface for an ultrasound imaging system including a rotatable cursor consistent with embodiments disclosed herein.

FIG. 13 illustrates another exemplary interface for an ultrasound imaging system including a user-defined region of interest consistent with embodiments disclosed herein.

FIG. 14 illustrates another exemplary interface for an ultrasound imaging system including a movable user-defined region of interest consistent with embodiments disclosed herein.

FIG. 15 illustrates another exemplary interface for an ultrasound imaging system including a scalable user-defined region of interest consistent with embodiments disclosed herein.

FIG. 16 illustrates another exemplary interface for an ultrasound imaging system including a scalable user-defined region of interest consistent with embodiments disclosed herein.

FIG. 17 illustrates another exemplary interface for an ultrasound imaging system including a user-defined region of interest consistent with embodiments disclosed herein.

FIG. 18 illustrates another exemplary interface for an ultrasound imaging system including a measurement system consistent with embodiments disclosed herein.

FIG. 19 illustrates another exemplary interface for an ultrasound imaging system including multi-segment tracing consistent with embodiments disclosed herein.

FIG. 20 illustrates an exemplary interface for an ultrasound imaging system including scaling consistent with embodiments disclosed herein.

FIG. 21 illustrates a block diagram of a computer system for implementing certain embodiments disclosed herein.

### DETAILED DESCRIPTION

A detailed description of systems and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

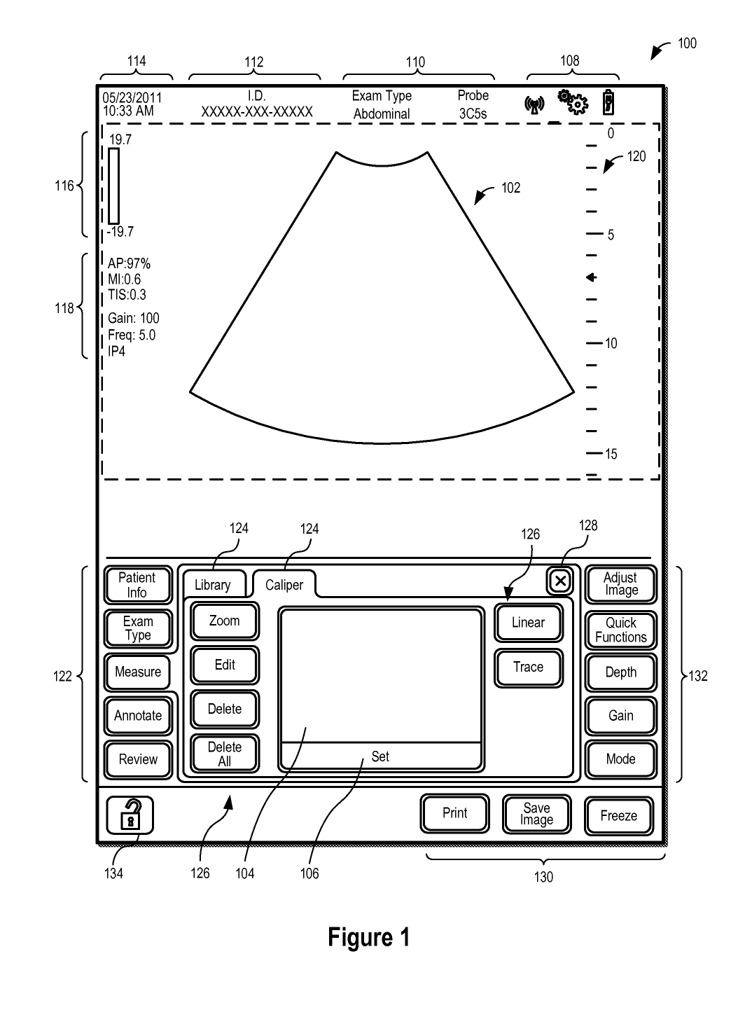

FIG. 1 illustrates an exemplary interface 100 for an ultrasound imaging system consistent with embodiments disclosed herein. Although embodiments disclosed herein are discussed in the context of a user interface for an ultrasound imaging system, embodiments may also be utilized in any other medical imaging and/or patient monitoring system. For example, embodiments may be utilized in a magnetic resonance imaging (“MRI”) system, a tomography system, a positron emission tomography (“PET”) system, and/or any other suitable medical imagining system.

As illustrated, the exemplary interface 100 may include a primary imaging area 102. The primary imaging area 102 may display images (e.g., real time or near-real time images) captured by the ultrasound imaging system. For example, images may be displayed in the primary imaging area 102 taken during an abdominal examination, a kidney examination, an early obstetrical examination, a late obstetrical examination, a gynecological examination, a thyroid examination, a breast examination, a testicular examination, an adult or pediatric cardiac examination, an upper or lower extremity arterial or venous vascular examination, a carotid vascular examination, and/or any other type of ultrasound imaging examination.

In certain embodiments, the interface 100 may be displayed on a touch screen panel that may be capable of detecting the presence and location of a touch (e.g., by a finger, hand, stylus, and/or the like) within the display area. The touch screen panel may implement any suitable type of touch screen technology including, for example, resistive touch screen technology, surface acoustic wave touch screen technology, capacitive touch screen technology, and/or the like. In certain embodiments, the touch screen panel may be a customized touch screen panel for the ultrasound imaging system. In further embodiments, the touch screen panel may be part of a discrete computing system incorporating a touch screen panel (e.g., an iPad or other suitable tablet computing device) configured to operate with the ultrasound imaging system.

A user may interact (i.e., provide input) with the touch screen panel and captured ultrasound images by touching the touch screen panel in relevant areas. For example, a user may touch the interface 100 within the primary imaging area 102 to interact with and/or control a displayed image. In certain embodiments, the interface 100 may include a touchpad 104. In some embodiments, a user's ability to interact with the interface 100 may be bounded within an area defined by the touchpad 104 and/or one or more function menus and buttons displayed on the interface 100. For example, a user may interact with the interface 100 within areas defined by the touchpad 104 and/or one or more function menus and not within other areas of the interface 100. Accordingly, if a user's finger crosses outside the area defined by the touchpad 104, the motion of the user's finger may not be utilized to interact with the primary imaging area 102 until the user's finger returns to the area defined by the touchpad 104. The touchpad 104 may further be configured to interact with and/or control any other area displayed on the interface 100.

A set button 106 may be disposed on the interface 100 proximate to the touchpad 104. The set button 106 may be used in conjunction with the touchpad 104 to interact with and/or control the ultrasound system. For example, a user may utilize the touchpad 104 to position a cursor over a particular area of the interface 100 and utilize the set button 106 to perform a certain function involving the area (e.g., selecting a particular function button and/or menu, placing a particular annotation and/or measurement marker, etc.) Alternatively, or in addition, a user may utilize the touchpad 104 to both position a cursor and to perform a certain function involving the cursor. For example, a user may utilize the touchpad 104 to position a cursor over a particular area of the interface 100 and also utilize the touchpad 104 (e.g., by tapping the touchpad twice or the like) to perform a certain function involving the area.

When interacting with the primary imaging area 102 and/or other areas displayed on the interface 100 using the touchpad 104, the user may utilize one or more functional tools. For example, a user may utilize the touchpad 104 to operate one or more marker tools, measurement tools, annotation tools, region of interests tools, and/or any other functional tools while interacting with the primary imaging area 102. Certain exemplary functional tools are described in more detail below.

In some embodiments, interacting with the touch screen panel via the touchpad 104 and/or one or more function menus and buttons may help to keep the primary imaging area 102 substantially clean from fingerprints, smudges, and/or any materials deposited by a user's fingers and hands. Interacting with the discrete touchpad 104 may also allow the user to interact with the primary imaging area 102 with a high degree of precision and without obscuring the primary imaging area 102. Further, utilizing a touch screen panel system may reduce mechanical malfunctions due to broken moving parts and may reduce the areas where contaminants may be deposited, thereby preserving the cleanliness of medical examination, operating, and/or hospital rooms.

The interface may include one or more system status indicators 108. In certain embodiments, the system status indicators 108 may include a power status indicator, a system configuration indicator, a network connectivity indicator, and/or any other type of system status indicator. The power status indicator may indicate whether the ultrasound system is coupled to AC power or, alternatively, powered by a battery. The system configuration indicator may indicate the status of certain system configurations. The network connectivity indicator may indicate the network connectivity status of the ultrasound system (e.g., connected via Wi-Fi). In certain embodiments, a user may access system status indicator sub-menus by touching any of the system status indicators 108 on the interface 100. For example, a user may touch the system configuration indicator and be presented with a sub-menu allowing the user to modify the configuration of the ultrasound system. Similarly, a user may touch the network connectivity indicator and be presented with a sub-menu allowing the user to view and/or modify the network connectivity of the ultrasound system.

The interface 100 may also display examination and probe type indicators 110. The examination indicator may indicate a type of examination being performed using the ultrasound system. For example, as illustrated, the examination indicator may indicate that the ultrasound system is being used to perform an abdominal examination. The probe type indicator may indicate a type of probe being used with the ultrasound system. In certain embodiments, a user may adjust the examination and/or probe type indicators 110 by touching the examination and/or probe type indicators 110 on the interface 100 and selecting an examination and/or probe type from the sub-menu displayed in response to the user's touch. In further embodiments, the ultrasound system may automatically detect an examination and/or probe type, and update the examination and probe type indicators 110 accordingly.

The interface 100 may further display patient identification information 112. In some embodiments, the patient identification information 112 may comprise a patient's name, gender, assigned identification number, and/or any other information that may be used to identify the patient. A user may adjust the patient identification 112 information by touching the patient identification information 112 on the interface 100 and entering appropriate patient identification information 112 into a sub-menu displayed in response to the user's touch. In certain embodiments, the patient identification information may be utilized to identify and access certain images captured by the ultrasound system.

A date and time indication 114 may further be displayed on the interface. In certain embodiments, the date and time indication 114 may be utilized to identify and access certain images captured by the ultrasound system (e.g., time-stamped images). A user may adjust the date and time information displayed in the date and time indication 114 by touching the date and time indication 114 on the interface 100 and entering appropriate date and time information into a sub-menu displayed in response to the user's touch.

Display scaling information 116 may be displayed on the interface 100 that provides information useful in viewing and/or interpreting ultrasound images displayed in the primary imaging area 102. For example, when ultrasound images displayed in the primary imaging area 102 are displayed in a grey scale format, the display scaling information 116 may provide an indication as to relative measurement degrees represented by each shade in the grey scale format. In embodiments where images displayed in the primary imaging area 102 are displayed in a color format, the display scaling information 116 may provide an indication as to relevant measurement degrees represented by each color in the color format. In certain embodiments, a user may adjust the display format of the images displayed in the primary imaging area 102 by touching the display scaling information 116 on the interface and selecting an appropriate display format in a sub-menu displayed in response to the user's touch.

The interface 100 may further display measurement parameter information 118. In certain embodiments, the measurement parameter information 118 may display measurement parameters associated with ultrasound images displayed in the primary imaging area 102. In some embodiments, the measurement parameter information 118 may be updated in real time or near real time with updates to the ultrasound images displayed in the primary imaging area 102. As illustrated, the measurement parameter information 118 may include an indication of AP, an indication of MI (e.g., acoustic power), an indication of the soft tissue thermal index (“TIS”), an indication of gain, and indication of frequency, and/or any other relevant measurement parameter information.

Primary imaging area scale information 120 may be displayed on the interface proximate to the primary imaging area 102. In certain embodiments, the primary imaging area scale information 120 may display a measurement scale that may assist a user in interpreting ultrasound images displayed in the primary imaging area 102. For example, using the primary imaging area scale information 120, a user may be able to determine a relative distance between two or more points included in an ultrasound image displayed in the primary imaging area 102. In further embodiments, the primary imaging area scale information 120 may include information related to a depth of view within a 3-dimensional image displayed in the primary imaging area. In certain embodiments, a user may adjust the relative scaling of the primary imaging area scale information 120 and/or the primary imaging area 102 by touching the primary imaging area scale information 120 on the interface 100 and selecting an appropriate relative scaling in a sub-menu displayed in response to the user's touch.

The interface 100 may include one or more top-level function menus 122. The top-level function menus 122 may provide one or more menu buttons defining one or more top-level functions a user may utilize to interact with and/or control the ultrasound imaging system. For example, as illustrated, the top-level function menus 122 may include a patient information menu button, an examination type menu button, a measure menu button, an annotate menu button, a review menu button, and/or menu buttons corresponding to any other type of top-level functions a user may wish to utilize.

In response to a user touching the patient information menu button, the user may be presented with a menu showing relevant patient information including, for example, patient identification information. Other relevant patient information may include patient history information, diagnosis information, and/or the like. In the patient information menu, the user may enter and/or adjust patient information as required. In response to the user touching the exam type menu button, the user may be presented with a menu relating to the particular exam type. In this menu, the user may enter and/or adjust examination type information. In certain embodiments, adjusting examination type information may result in a corresponding adjustment of operating parameters and/or settings for the ultrasound imaging system to optimize system performance for a particular examination type.

In response to a user touching the review menu button, the user may be presented with a menu allowing the user to review, organize, and/or interact with previously captured images. In certain embodiments, these previously captured images may be still ultrasound images. In further embodiments, these previously captured images may be moving ultrasound images. In response to touching the measure menu button, the user may be presented with a menu related to certain measurement functions, described in more detail below. Similarly, in response to touching the annotate measure button, the user may be presented with a menu relating to certain annotation functions, also described in more detail below.

After touching one of the top-level function menus 122, a user may be presented with a sub-menu that, in certain embodiments, may include one or more sub-level function menus 124. In certain embodiments, the one or more sub-level function menus 124 may relate to one or more sub-level functions associated with a selected top-level function menu 122. For example, as illustrated, when a user touches the measure menu button, a sub-menu that includes a library sub-level function menu and a caliper sub-level function menu may be presented. In certain embodiments, the library sub-level function menu may include one or more predefined measurement functional tools that a user may utilize to interact with and/or interpret images displayed in the primary imaging area 102.

In certain embodiments, after touching one of the sub-level function menus 124, the user may be presented with one or more associated function buttons 126 allowing the user to perform certain functions associated with the function buttons 126. For example, as illustrated, when a user touches the caliper sub-level function menu, associated function buttons 126 including a zoom button, an edit button, a delete button, a delete all button, a linear button, a trace button, and/or any other related function button may be presented. When the zoom button is touched, a user may perform zooming operations on the images displayed in the primary imaging area 102. In certain embodiments, zooming operations may be performed using the touchpad 104. For example, a user may utilize a “spread” gesture (i.e., drawing two fingers on the touchpad 104 apart) to perform a zooming operation on an image displayed in the primary imaging area 102. Any other suitable gesture using one or more contact points on the touchpad 104 may also be utilized to perform zooming operations.

When the linear button is touched, a user may be presented with a cursor that may be used to perform linear measurement of the image displayed in the primary imaging area 102. Similarly, when the trace button is touched, a user may be presented with a tracing cursor for performing a multi-segment measurement of the image displayed in the primary imaging area 102. If a user wishes to change certain markers utilized in measurements, the user may touch the edit button, thereby allowing them to reposition the markers relative to the image displayed in the primary imaging area 102 using, for example, the touchpad 104. If a user wishes to delete a particular marker utilized in measurements, the user may touch the delete button, thereby allowing them to delete the particular marker using, in some instances, the touchpad. Similarly, if a user wishes to delete all markers utilized in measurements, the user may touch the delete all button.

Depending on the selected top-level function menu 122, the touchpad 104 may be displayed as part of the sub-menu associated with the top-level function menu 122. For example, as illustrated in FIG. 1, the touchpad 104 and/or set button 106 may be displayed in a sub-menu as part of the caliper sub-level function menu of the sub-level function menus 124. When a user is finished utilizing operations and/or functions associated with a particular sub-menu, the user may touch a close button 128 to close the sub-menu. If a user wishes to later reopen a particular sub-menu, the user may touch the corresponding top-level function menu 122.

The interface 100 may further include one or more image capture buttons 130 that may be utilized to capture certain still and/or moving images displayed in the primary imaging area 102. As illustrated, the one or more capture buttons 130 may include a print button, a save button, and a freeze button. Touching the print button may print a copy of one or more images displayed in the primary imaging area 102. In certain embodiments, touching the print button may open a print sub-menu that the user may utilize to control printer settings and print a copy of the one or more images. Touching the save button may save a copy of one or more moving and/or still images displayed in the primary imaging area 102. In certain embodiments, touching the save button may open up a save sub-menu that the user may utilize to control image saving properties. Touching the freeze button may cause a certain still image or frame of a moving image displayed in the primary imaging area 102 to freeze, thereby allowing a user to study the frozen image in more detail.

One or more display function buttons 132 may be included on the interface 100. For example, as illustrated, an adjust image button, a quick function button, a depth function button, a gain button, and/or a mode button may be included on the interface. Touching the adjust image button may open up a menu allowing the user to make one or more adjustments to images displayed in the primary imaging area 102. Touching the quick function button may open up a menu allowing the user to select one or more functions and/or operations that may be used in controlling, viewing, and/or interpreting images displayed in the primary imaging area 102. Touching the depth button may allow a user to adjust a depth of view within a 3-dimensional image displayed in the primary imaging area 102. For example, in certain embodiments a “pinch” gesture using two fingers on the touchpad 104 may adjust a depth of view within a 3-dimensional medical image displayed in the primary imaging area 102. Touching the gain button may open up a menu that allows a user to adjust a gain of the ultrasound imaging system. Finally, touching the mode button may open up a menu that allows a user to adjust an operating mode of the ultrasound imaging system.

In certain embodiments, a user may wish to prevent inadvertent input from being provided to the interface 100. Accordingly, a user may touch a screen lock button 134 configured to cause the interface 100 to lock, thereby preventing a user from providing input by inadvertently touching the interface 100. If a user wishes to restore functionality to the interface 100, the user may touch the screen lock button again, thereby unlocking the interface 100.

It will be appreciated that a number of variations can be made to the architecture, relationships, and functions presented in connection with FIG. 1 within the scope of the inventive body of work. For example, certain interface 100 layouts, architectures, and functionalities may be arranged and/or configured in any suitable manner within the scope of the inventive body of work. Further, certain functionalities using the touchpad 104 may be implemented utilizing any suitable gestures and/or number of contact points. Thus, it will be appreciated that the interface 100 of FIG. 1 is provided for purposes of illustration and explanation, and not limitation.

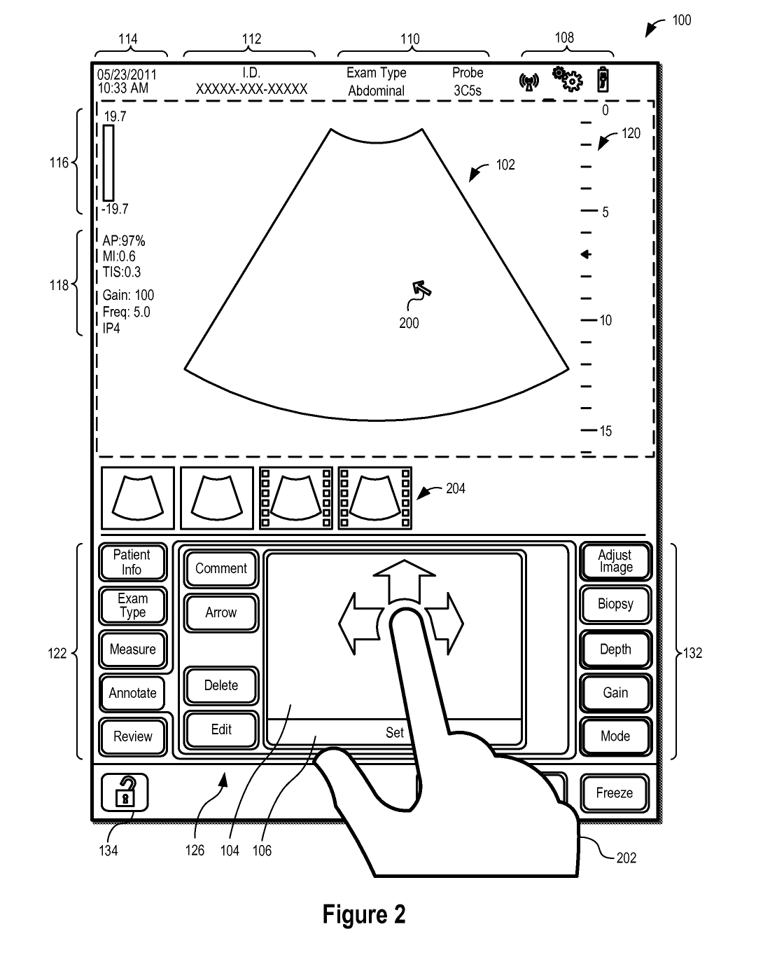

FIG. 2 illustrates an exemplary interface 100 for an ultrasound imaging system including a cursor 200 consistent with embodiments disclosed herein. Certain elements of the exemplary interface 100 may be similar to those illustrated and described in reference to FIG. 1, and, accordingly, similar elements may be denoted with like numerals. As illustrated and discussed above, in interacting with the interface 100, a user 202 may touch a displayed touchpad 104. In various functions and operations utilizing the interface 100, the relative movement of a user's 202 finger on the touchpad 104 may cause a cursor 200 to move accordingly. For example, a user 202 may cause the cursor 200 to move in a right-direction by moving their finger in a right-direction on the track pad.

In certain embodiments, the cursor 200 may be utilized in certain annotation functions and/or operations associated with the aforementioned annotate menu button of the top-level function menus 122. As illustrated, the annotate menu button may be associated with one or more function buttons 126 including a comment button, an arrow button, a delete button, and an edit button. When a user 202 touches the comment button, a menu may be displayed that allows the user 202 to enter a comment associated with the image displayed in the primary imaging area 102. In certain embodiments, the menu may include a touch screen keyboard allowing the user 202 to enter the comment. The comment may be associated with a particular portion of the image displayed in the primary imaging area 102 or, alternatively, the entire image. In embodiments where the comment is associated with a portion of the image, a flag, cross, arrow, or similar annotation may be placed on the particular portion of the image. In embodiments where the comment is associated with the entire image, an indication that there is a comment associated with the image may be displayed on the interface 100. Further, the comment and/or any other annotations disclosed herein may be included in any saved copy of the image.

When a user 202 touches the arrow button, the user 202 may annotate the image displayed in the primary imaging area 102 by placing an arrow or other marker over the image. For example, after touching the arrow button, the user 202 may position an arrow over the image displayed in the primary imaging area 102 by touching the primary imaging area 102 and/or by utilizing the touchpad 104. After positioning the arrow in a desired location, the user 202 may place the arrow over the image by touching the set button 106 and/or touching the primary imaging area 102 in a manner that places the arrow in the particular location (e.g., double tapping the primary imaging area 102 at the desired location).

When a user 202 touches the delete button, the user 202 may position the cursor 200 over an annotation or comment made in the primary imaging area 102 by touching the primary imaging area 102 at the annotation or comment and/or by utilizing the touch pad 104. The user 202 may delete the annotation by either touching the set button 106 or by touching the primary imaging area 102 in a manner that deletes the annotation (e.g., double tapping the primary imaging area 102 at the location of the annotation).

When a user 202 touches the edit button, the user 202 may position the cursor 200 over an annotation or comment made in the primary imaging area 102 by touching the primary imaging area 102 at the annotation or comment and/or by utilizing the touch pad 104. The user may then select the annotation or comment for editing by either touching the set button 106 to open up an editing menu or by touching the primary imaging area 102 in a manner that opens up an editing menu for the selected annotation or comment. In certain embodiments, the editing menu may include a touch screen keyboard allowing the user 202 to edit the comment and/or annotation as desired.

A menu button may be provided for certain common functions and/or annotation operations that, in certain embodiments, may be dependent on a selected examination type. For example, as illustrated, marking an area of the image displayed in the primary imaging area 102 for a future biopsy may be common. Accordingly, a menu button for a biopsy annotation may be displayed in the interface 100, thereby streamlining the ability of a user 202 to make such an annotation.

FIG. 2 further illustrates one or more captured ultrasound images 204 displayed on the interface 100. As discussed above in reference to FIG. 1, in certain embodiments, a user 202 may save a copy of one or more moving and/or still images displayed in the primary imaging area 102. In certain embodiments, when a copy of a still or a moving image is saved, a preview image may be displayed of the saved images as one or more captured ultrasound images 204. In certain embodiments, when the captured ultrasound image 204 is a still image, the displayed preview image may be a smaller copy of the corresponding saved image. Similarly, when the captured ultrasound image 204 is a moving image, the displayed preview image may be a single frame of the corresponding saved moving image and/or may include an indication that the captured ultrasound image 204 is a moving image. When a user touches any of the one or more captured ultrasound images 204, the corresponding still or moving captured ultrasound images 204 may be displayed in the primary imaging area 102.

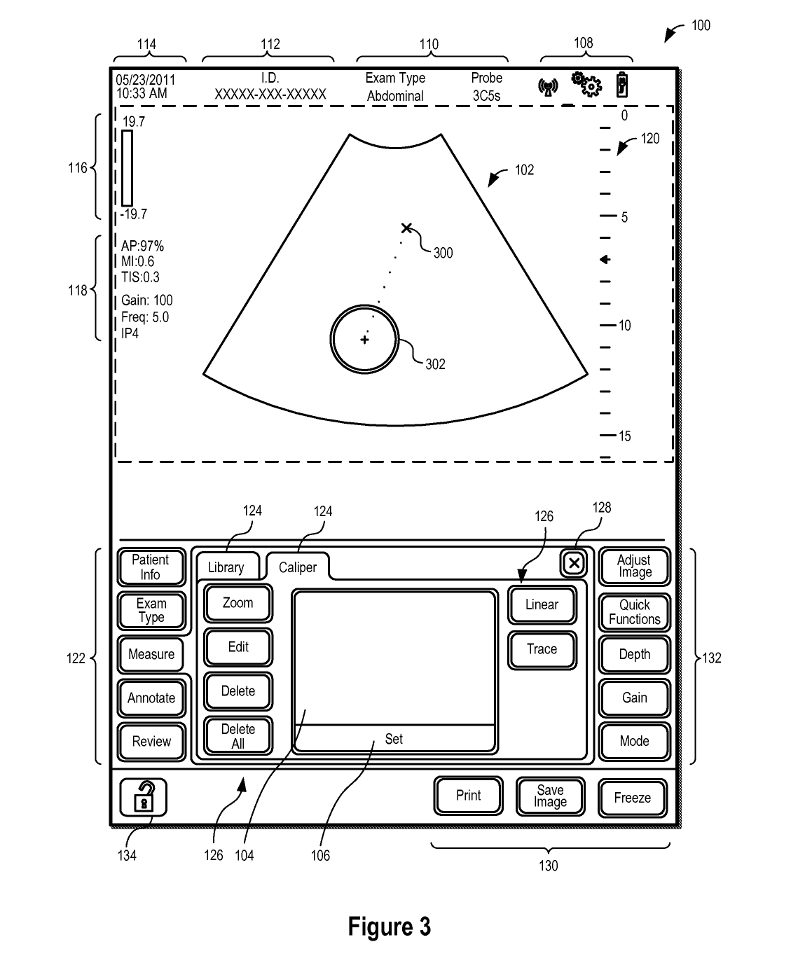

FIG. 3 illustrates an exemplary interface 100 for an ultrasound imaging system including an off-set cursor 300 consistent with embodiments disclosed herein. Certain elements of the exemplary interface 100 may be similar to those illustrated and described in reference to FIGS. 1-2, and, accordingly, similar elements may be denoted with like numerals. In certain circumstances, a user 202 may wish to interact directly with the images displayed in the primary imaging area 102 of the interface 100 rather than utilizing the touchpad 104. Interacting with (e.g., touching) an area of interest of an image displayed in the primary imaging area 102, however, may result in the user 202 obscuring the area of interest with their hands and/or fingers. Accordingly, in some embodiments, the interface 100 may utilize a touch area 302 that is off-set from a cursor 300.

A user 202 may touch the interface 100 at the touch area 302, which in certain embodiments, may be positioned anywhere on the interface 100. At a particular distance and orientation from the touch area 302, an off-set cursor 300 may appear. When the user 202 moves the position of where they are touching the interface 100 (i.e., the touch area 302), their movements may be translated into a corresponding movement in the off-set cursor 300. In this manner, a user 202 may precisely move the off-set cursor 300 as desired while maintaining a clear view of the interface 100 and/or primary imaging area 102.

As illustrated, in some embodiments, a line (e.g., a dotted line) may be displayed between the touch area 302 and the off-set cursor 300, thereby aiding a user 202 in identifying the relative position of the off-set cursor 300 with respect to the touch area 302. Moreover, a user 202 may utilize the touch area 302 to interact with the interface 100 using single-point touch screen commands. Further, in certain embodiments, a user 202 may utilize a plurality of touch areas 302 and/or off-set cursors 300 to interact with the interface 100 using any number of multi-point gesture commands. For example, a user 202 may zoom into an image displayed in the primary imaging area 102 defined by two off-set cursors 300 by moving the two respective touch points 302 associated with the off-set cursors 300 apart in a “spread” gesture. The touch area 302 may be similarly utilized to select an item displayed on the interface under an off-set cursor 300 (e.g., by tapping the touch area 302 twice or the like).

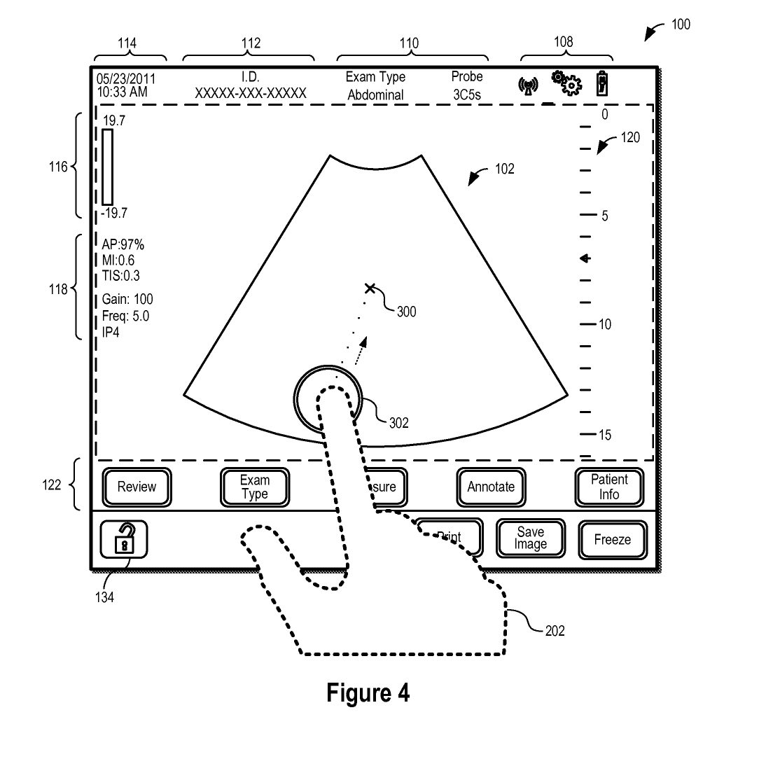

FIG. 4 illustrates another exemplary interface 100 for an ultrasound imaging system including an off-set cursor 300 consistent with embodiments disclosed herein. Certain elements of the exemplary interface 100 may be similar to those illustrated and described in reference to FIGS. 1-3, and, accordingly, similar elements may be denoted with like numerals. As discussed above, a user 202 may wish to interact directly with the images displayed in the primary imaging area 102 of the interface 100. Interacting with (e.g., touching) an area of interest of an image displayed in the primary imaging area 102, however, may result in the user 202 obscuring the area of interest with their hands and/or fingers. Moreover, interacting with an area of interest directly may result in less precise control of a cursor, annotation, measurement marker point, or the like.

As illustrated, the interface 100 may utilize a touch area 302 within the primary imaging area 102 that is off-set from a cursor 300. In certain embodiments utilizing a touch area 302 and an off-set cursor 300 within the primary imaging area 102, the interface 100 may not include a touchpad area as discussed above in reference to FIGS. 1-3. At a particular distance and orientation from the touch area 302, an off-set cursor 300 may appear. When the user 202 moves the position of where they are touching the interface 100 (i.e., the touch area 302), the user's movements (in the direction of the arrow) may be translated into a corresponding movement in the off-set cursor 300. In this manner, a user 202 may precisely move the off-set cursor 300 as desired while maintaining a clear view of the interface 100 and/or primary imaging area 102. In certain embodiments, off-set positioning of a touch area 302 and an area of interest (e.g., an off-set cursor 300) may be utilized in annotation operations, commenting operations, measuring operations, and/or any other interface 100 operations and/or functionalities described herein.

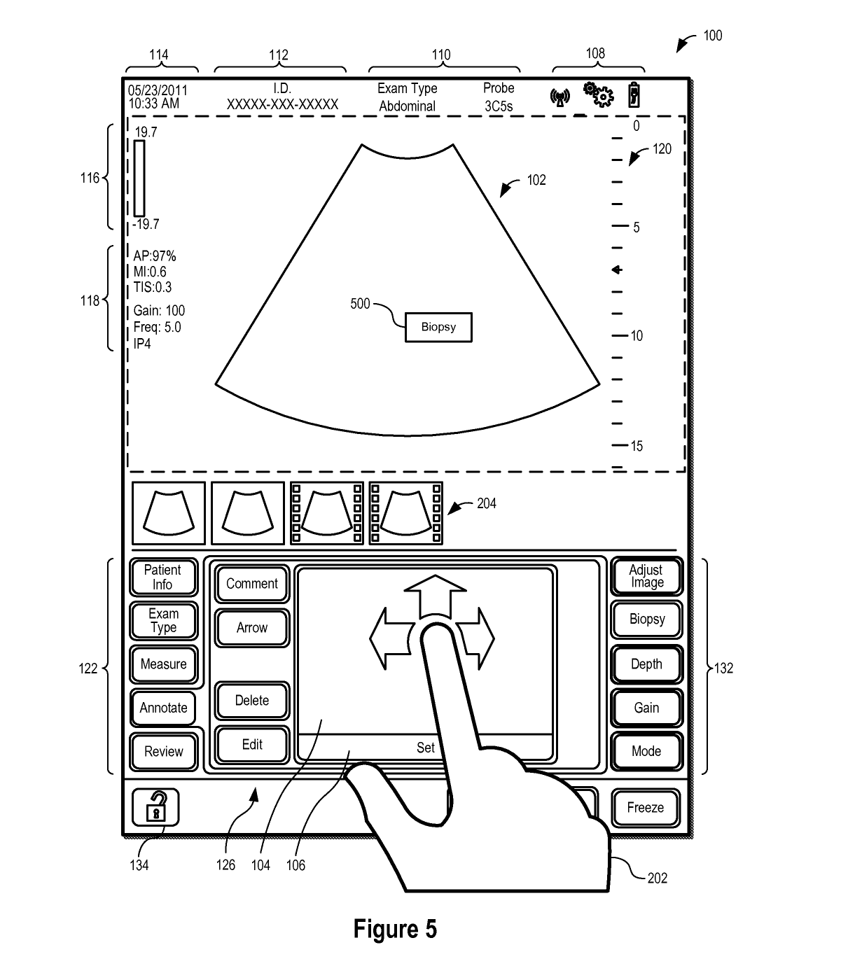

FIG. 5 illustrates an exemplary interface 100 for an ultrasound imaging system including an annotation 500 consistent with embodiments disclosed herein. Certain elements of the exemplary interface 100 may be similar to those illustrated and described in reference to FIGS. 1-4, and, accordingly, similar elements may be denoted with like numerals. As described above, the interface 100 may allow a user 202 to annotate and/or comment on an image displayed in the primary imaging area 102. For example, a user 202 may wish to mark a certain area of a displayed image for a future biopsy. Accordingly, as illustrated, using the annotate menu and the touchpad 104, the user 202 may position an annotation 500 marking an area of an image displayed in the primary imaging area 102 for biopsy. In some embodiments, the user may place the annotation 500 by touching the set button 106. In further embodiments, the user may position the annotation 500 by touching the interface 100 on or near the area on the image displayed in the primary imaging area 102 (e.g., using the off-set cursor 300 discussed in reference to FIG. 3), and place the annotation 500 by tapping the interface 100 twice and/or touching the set button 106.

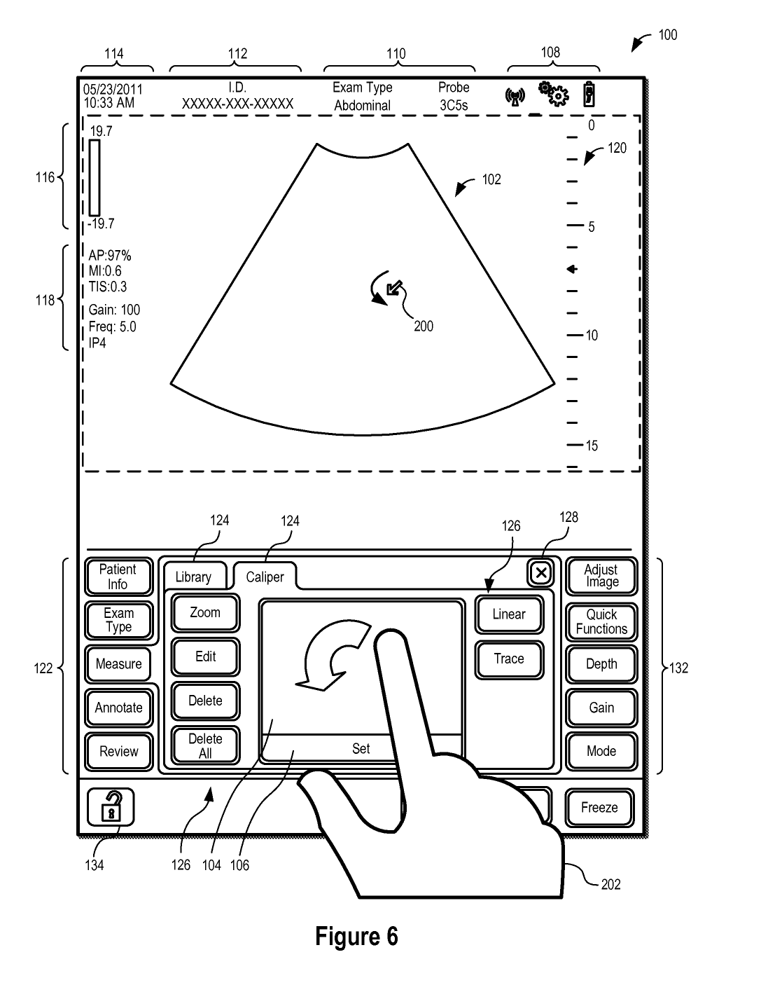

FIG. 6 illustrates an exemplary interface 100 for an ultrasound imaging system including a rotatable cursor consistent with embodiments disclosed herein. Certain elements of the exemplary interface 100 may be similar to those illustrated and described in reference to FIGS. 1-5, and, accordingly, similar elements may be denoted with like numerals. As discussed above in reference to FIG. 2, a user 202 may utilize a cursor 200 to interact with, comment, and/or annotate images displayed in the primary imaging area 102. In certain embodiments, a user 202 may wish to rotate the orientation of a cursor 200, comment, and/or annotation (e.g., an arrow, marker, or the like). To facilitate such rotation of a cursor 200, comment, and/or annotation, a user 202 may utilize a suitable gesture using one or more contact points on touchpad 104. For example, a user may place the cursor 200, comment, and/or annotation in a desired position within the primary imaging area 102 and, as illustrated, may rotate the cursor 200, comment, and/or annotation by using a “rotate” gesture with one or more contact points on the touchpad 104. Any other suitable gesture using one or more contact points on the touchpad 104 may also be utilized to perform rotating and/or positioning operations. Further, suitable gestures may be utilized using one or more contact points on areas of the interface 100 other than the touchpad 104 (e.g., at or near the desired position of the cursor 200, comment, and/or annotation).

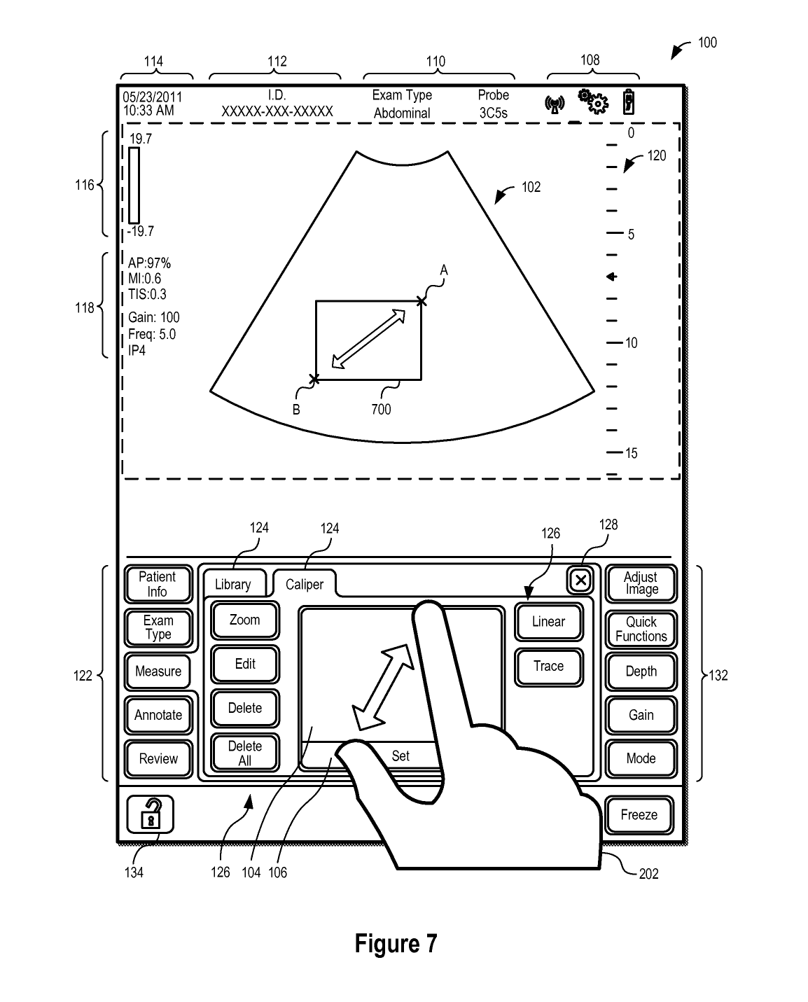

FIG. 7 illustrates an exemplary interface 100 for an ultrasound imaging system including a user-defined region of interest consistent with embodiments disclosed herein. Certain elements of the exemplary interface 100 may be similar to those illustrated and described in reference to FIGS. 1-6, and, accordingly, similar elements may be denoted with like numerals. In certain embodiments, a user 202 may wish to define a region of interest 700 within an image displayed in the primary imaging area 102. In certain embodiments, a region of interest 700 may be an area that the user 202 wishes to view in higher magnification, an area that the user 202 wishes to measure, an area that the user 202 wishes to annotate for later study in detail, and/or any other desired interest.

To define a region of interest 700, the user 202 may touch the touchpad 104 at a plurality of contact points. For example, as illustrated, the user 202 may touch the touchpad 104 at two contact points. The user 202 may then define a region of interest 700 by utilizing a “spread” gesture on the touchpad 104 (i.e., by drawing two fingers on the touchpad 104 apart to points “A” and “B” as illustrated). In embodiments where two contact points are utilized, the region of interest 700 may be defined by a square or rectangle having opposing corners at the two contact points. Any other suitable number of contact points, region of interest shapes, and/or gestures may also be utilized to define a region of interest 700.

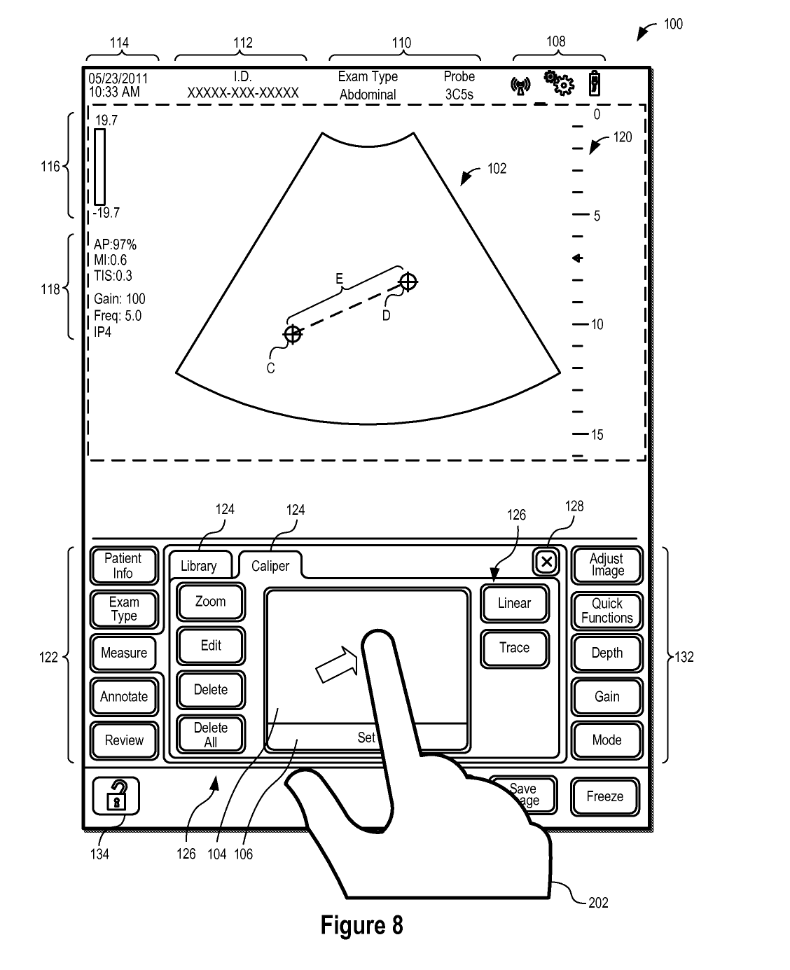

FIG. 8 illustrates an exemplary interface 100 for an ultrasound imaging system including a measurement system consistent with embodiments disclosed herein. Certain elements of the exemplary interface 100 may be similar to those illustrated and described in reference to FIGS. 1-7, and, accordingly, similar elements may be denoted with like numerals. As discussed above, a user 202 may utilize measurement functions accessed via a measure menu button that may allow the user 202 to measure certain portions of images displayed in the primary imaging area 102.

In certain embodiments, a user 202 may measure images displayed in the primary imaging area 102 by defining one or more measurement marker points within the displayed images. For example, as illustrated, a user 202 may define a first measurement marker point “C” within the primary imaging area 102. In certain embodiments, the first measurement marker point may be defined by positioning the measurement marker point “C” in a particular location in the primary imaging area 102 using the touchpad 104 and/or by touching the primary imaging area 102 directly. The user 202 may place the measurement marker point “C” by touching the set button 106 and/or by using an appropriate gesture (e.g., a double tap at the location) on the primary imaging area 102. The user 202 may then define a second measurement marker point “D” within the primary imaging area 102 by positioning the measurement marker point “D” in a particular location in the primary imaging area 102 using the touchpad 104 and/or by touching the primary imaging area 102 directly. The user 202 may place the measurement marker point “D” by touching the set button 106 and/or by using an appropriate gesture on the primary imaging area 102. The interface 100 may then display a measurement “E” indicating the relative distance between the measurement marker point “C” and measurement marker point “D.”

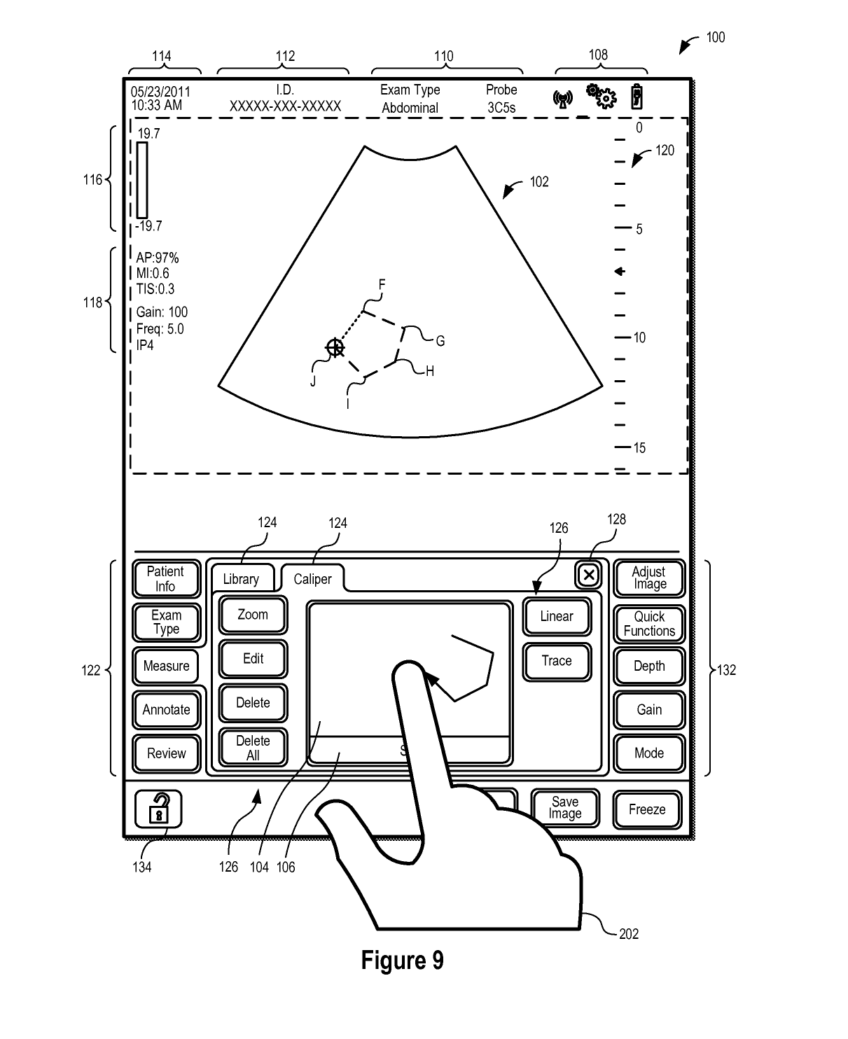

FIG. 9 illustrates an exemplary interface 100 for an ultrasound imaging system including multi-segment tracing consistent with embodiments disclosed herein. Certain elements of the exemplary interface 100 may be similar to those illustrated and described in reference to FIGS. 1-8, and, accordingly, similar elements may be denoted with like numerals. As discussed above, a user 202 may utilize tracing functions to perform multi-segment measurements of an image displayed in the primary imaging area 102. In certain embodiments, a multi-segment trace may be performed by placing one or more measurement marker points (e.g., measurement marker points “F”, “G”, “H”, “I”, and “J”) at particular locations in the primary imaging area 102. A tracing path may be defined having vertices corresponding to the measurement marker points. In certain embodiments, the interface 100 may be configured to automatically finalize a final segment of a tracing path by creating a segment between the first placed measurement marker point (e.g., point “F”) and a last placed measurement marker point (e.g., point “J”).

In some embodiments, the multi-segment trace path may be used for measurement purposes. For example, a measurement length of the multi-segment trace path may be displayed in the interface 100. In further embodiments, the multi-segment trace path may be utilized in zooming operations, in annotation operations, and/or the like.

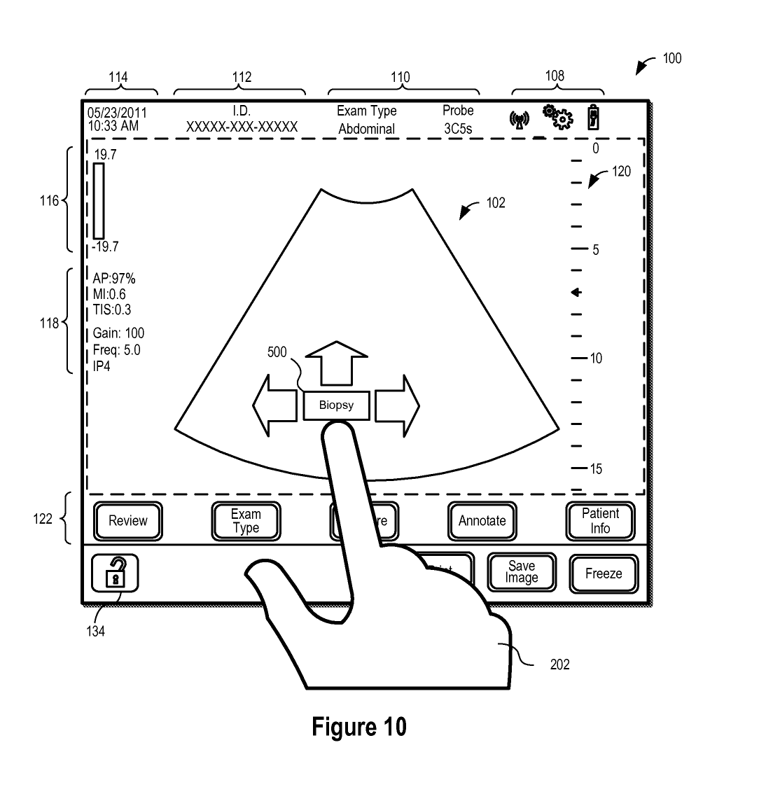

FIG. 10 illustrates another exemplary interface 100 for an ultrasound imaging system including an annotation 500 consistent with embodiments disclosed herein. Certain elements of the exemplary interface 100 may be similar to those illustrated and described in reference to FIGS. 1-9, and, accordingly, similar elements may be denoted with like numerals. As described above, the interface 100 may allow a user 202 to annotate and/or comment on an image displayed in the primary imaging area 102. A user 202 may wish to annotate and/or comment on an image displayed in the primary imaging area 102 by interacting directly with the images (e.g., touching the images) displayed in the primary imaging area 102. For example, a user 202 may wish to mark a certain area of a displayed image for a future biopsy. Accordingly, as illustrated, the user 202 may position an annotation 500 by touching an area of an image displayed in the primary imaging area 102 and moving the area to a desired location to annotate for a biopsy. The user 202 may further place the annotation 500 by tapping the primary imaging area 102 in a particular area (e.g., a desired annotation location), releasing their touch on the primary imaging area 102 when the annotation 500 is in a desired location, or any other suitable touch operation.

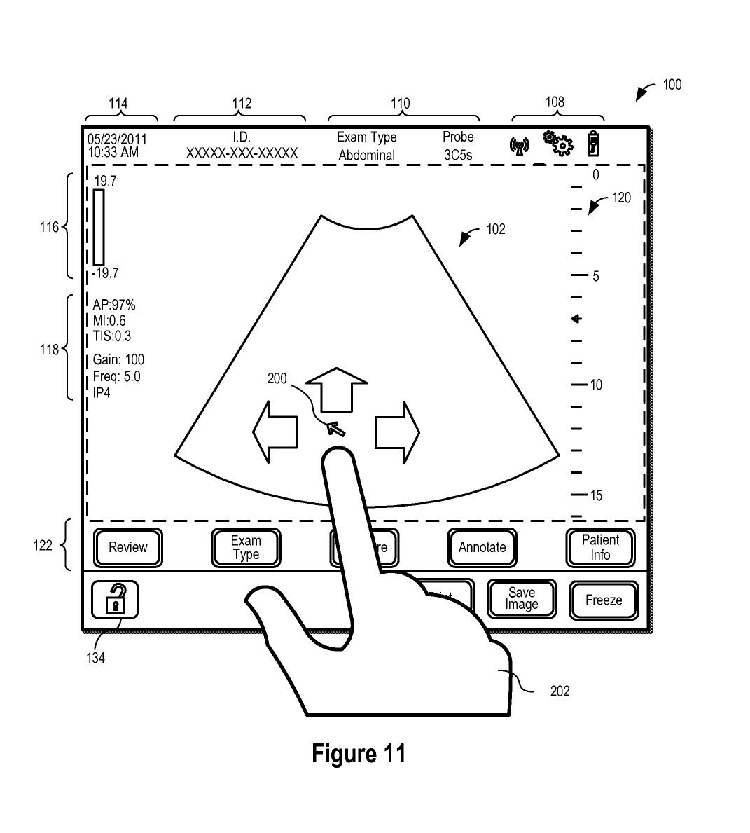

FIG. 11 illustrates another exemplary interface 100 for an ultrasound imaging system including a cursor 200 consistent with embodiments disclosed herein. Certain elements of the exemplary interface 100 may be similar to those illustrated and described in reference to FIGS. 1-10, and, accordingly, similar elements may be denoted with like numerals. As discussed above, a user 202 may utilize a cursor 200 to interact with the interface 100. As discussed above, in various functions and operations utilizing the interface 100, the relative movement of a user's 202 finger on the interface 100 may cause a cursor 200 displayed on the interface 100 to move accordingly. For example, a user 202 may wish to interact with the primary imaging area 102 of the interface 100. The user 202 may then touch the primary imaging area 102 in a certain area and a cursor 200 may appear at the area. The user 202 may then move the cursor 200 by moving the relative position of the area. For example, a user 202 may cause the cursor 200 to move in a right-direction by moving their finger in a right-direction while touching the primary imaging area 102.

In certain embodiments, after positioning the cursor 200, a user 202 may wish to place the cursor 200 in a particular location. The user 202 may place the cursor 200 by tapping the primary imaging area 102 in a particular area (e.g., a desired cursor location), releasing their touch on the primary imaging area 102 when the cursor 200 is in a desired location, and/or by using any other suitable touch operation.

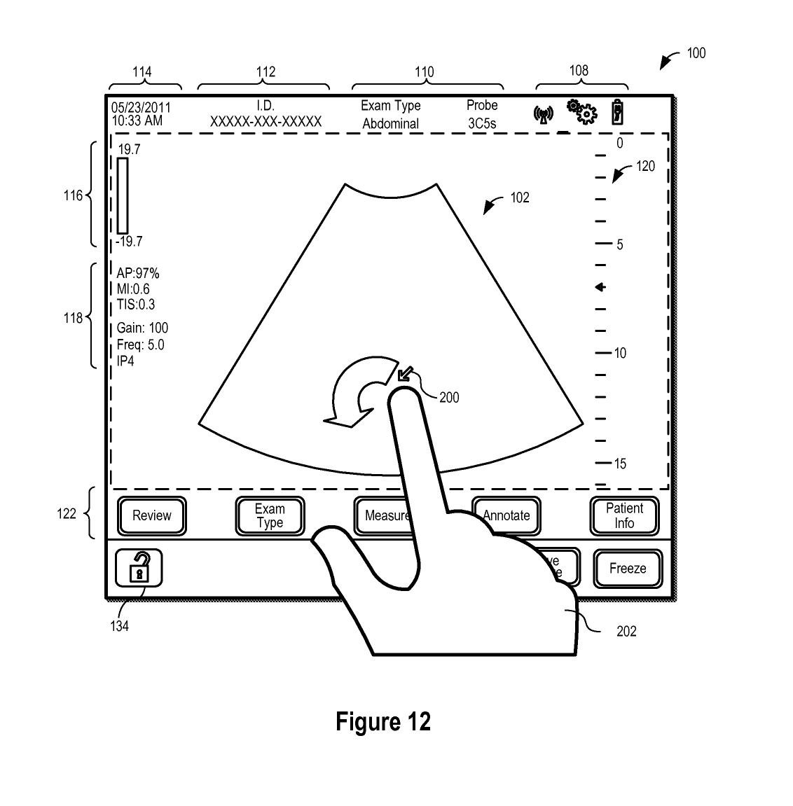

FIG. 12 illustrates another exemplary interface 100 for an ultrasound imaging system including a rotatable cursor 200 consistent with embodiments disclosed herein. Certain elements of the exemplary interface 100 may be similar to those illustrated and described in reference to FIGS. 1-11, and, accordingly, similar elements may be denoted with like numerals. As discussed above, a user 202 may utilize a cursor 200 to interact with, comment, and/or annotate images displayed in a primary imaging area 102 of the interface 100. In certain embodiments, a user 202 may wish to rotate the orientation of a cursor 200, comment, and/or annotation (e.g., an arrow, marker, or the like) while interacting directly with the primary imaging area 102. To facilitate such rotation of a cursor 200, comment, and/or annotation, a user 202 may utilize a suitable gesture using one or more contact points on interface 100 (e.g., on the primary imaging area 102). For example, a user may place the cursor 200, comment, and/or annotation in a desired position within the primary imaging area 102 and, as illustrated, may rotate the cursor 200, comment, and/or annotation by using a “rotate” gesture with one or more contact points on the interface 100. Any other suitable gesture using one or more contact points on the interface 100 may also be utilized to perform rotating and/or positioning operations.

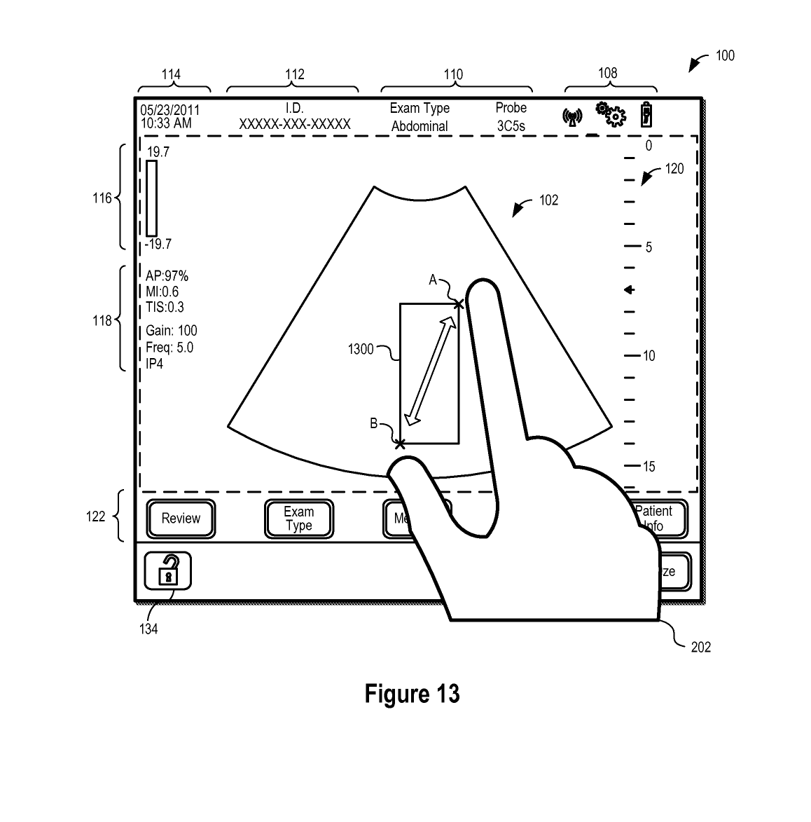

FIG. 13 illustrates another exemplary interface 100 for an ultrasound imaging system including a user-defined region of interest 1300 consistent with embodiments disclosed herein. Certain elements of the exemplary interface 100 may be similar to those illustrated and described in reference to FIGS. 1-12, and, accordingly, similar elements may be denoted with like numerals. As discussed above, a user 202 may wish to define a region of interest 1300 within an image displayed in the primary imaging area 102. A region of interest 300 may be an area that the user 202 wishes to view in higher magnification, an area that the user 202 wishes to measure, an area that the user 202 wishes to annotate for later study in detail, and/or interests the user 202 in any other way.

To define a region of interest 1300, the user 202 may interact directly with images displayed in the primary imaging area 102 by touching the interface 100 at a plurality of contact points within the primary imaging area 102. For example, as illustrated the user 202 may touch the interface 100 at two contact points. The user 202 may then define a region of interest 1300 by utilizing a “spread” gesture on the interface 100 (e.g., by drawing two or more fingers apart while contacting the primary imaging area 102). In embodiments where two contact points are utilized, the region of interest 1300 may be defined by a square or rectangle having opposing corners at the contact points. Any other suitable number of contact points, region of interest shapes, and/or gestures may also be utilized to define a region of interest 1300.

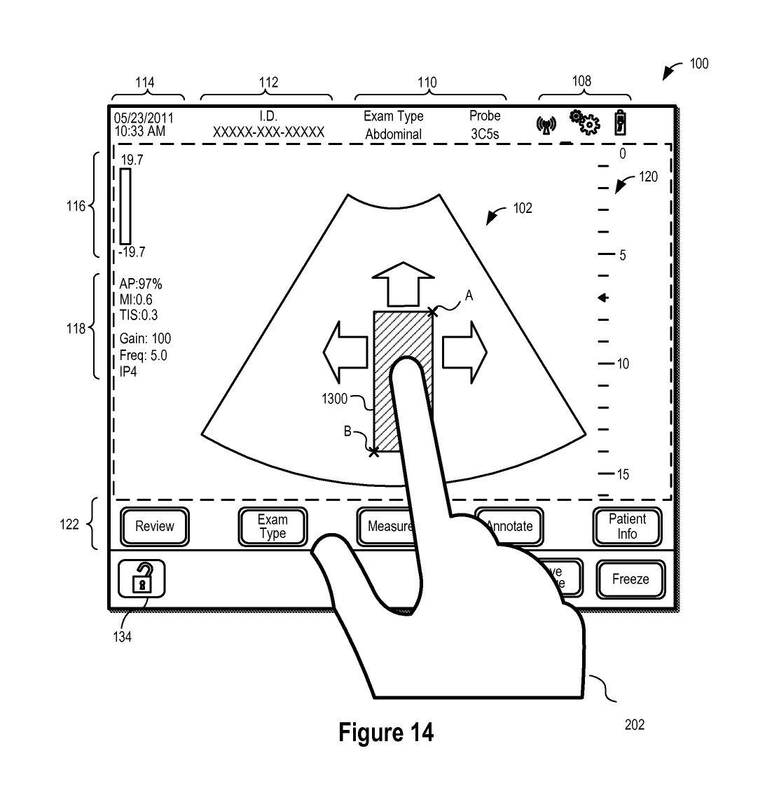

FIG. 14 illustrates another exemplary interface 100 for an ultrasound imaging system including a movable user-defined region of interest 1300 consistent with embodiments disclosed herein. Certain elements of the exemplary interface 100 may be similar to those illustrated and described in reference to FIGS. 1-13, and, accordingly, similar elements may be denoted with like numerals. In certain circumstances, a user 202 may wish to reposition and/or move a previously defined region of interest 1300. To move the region of interest 1300, a user may first select the region of interest 1300 by touching and holding the area of the interface 100 corresponding to the region of interest 1300, by tapping the area of the interface 100 corresponding to the region of interest 1300 twice, and/or by any other suitable touch input for selecting the region of interest 1300. Once selected, the region of interest 1300 may be moved by moving the relative position of their contact point on the interface 100. For example, a user 202 may cause the region of interest 1300 to move in a right-direction by moving their finger in a right-direction while touching an area of the primary imaging area 102 corresponding to the region of interest 1300.

In certain embodiments, after positioning the region of interest 1300, a user 202 may wish to place the region of interest 1300 in a particular location within the primary imaging area 102. The user 202 may place the region of interest 1300 by tapping the primary imaging area 102 in a particular area (e.g., a desired cursor location), releasing their touch on the primary imaging area 102 when the region of interest 1300 is in a desired location, and/or by using any other suitable touch operation.

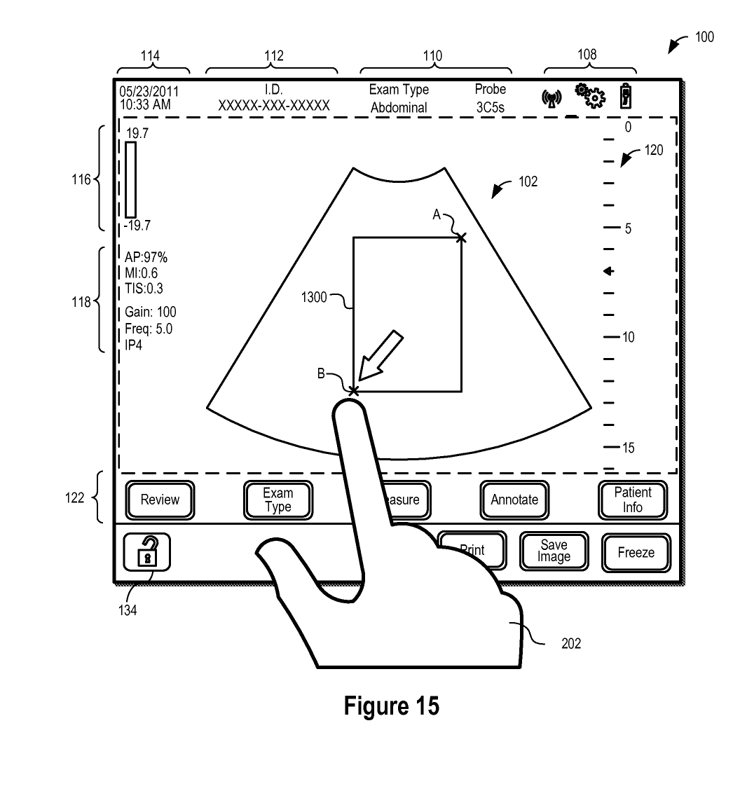

FIG. 15 illustrates another exemplary interface 100 for an ultrasound imaging system including a scalable user-defined region of interest 1300 consistent with embodiments disclosed herein. Certain elements of the exemplary interface 100 may be similar to those illustrated and described in reference to FIGS. 1-14, and, accordingly, similar elements may be denoted with like numerals. In certain circumstances, a user 202 may wish to resize and/or scale a previously defined region of interest 1300. To resize and/or scale a previously defined region of interest 1300, a user 202 may touch one or more of the corners of the region of interest 1300 (e.g., at point “B” as illustrated) and change the position of the one or more corners, thereby causing the area defined by the region of interest 1300 to change. For example, as illustrated, a user may “pull” a corner of the region of interest 1300 outwards, thereby increasing the area of the region of interest 1300.

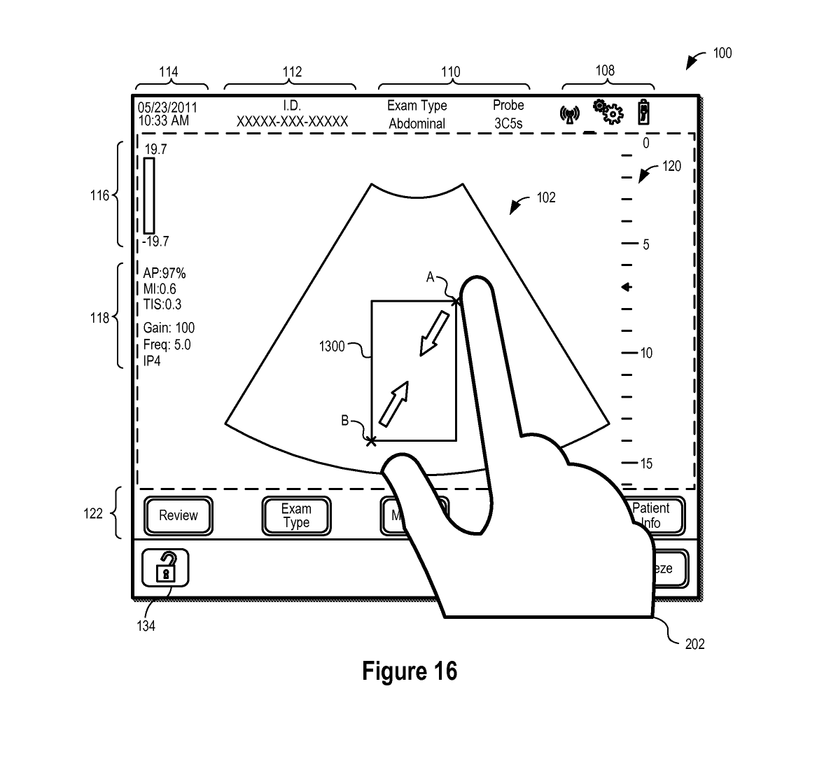

FIG. 16 illustrates another exemplary interface for an ultrasound imaging system including a scalable user-defined region of interest consistent with embodiments disclosed herein. Certain elements of the exemplary interface 100 may be similar to those illustrated and described in reference to FIGS. 1-15, and, accordingly, similar elements may be denoted with like numerals. As noted above, a user 202 may wish to resize and/or scale a previously defined region of interest 1300. For certain resizing and/or scaling operations, a user 202 may utilize a plurality of touch contact points on the interface 100 to resize and/or scale a previously defined region of interest 1300. For example, as illustrated, the user 202 may utilize a “pinch” gesture by pulling two fingers contacting opposite corners of the region of interest 1300 together to make the region of interest 1300 smaller. Similarly, a user 202 may utilize a “spread” gesture by spreading two fingers contacting opposite corners of the region of interest 1300 apart to make the region of interest 1300 larger. Any other suitable gesture may also be used for resizing and/or scaling the region of interest 1300.

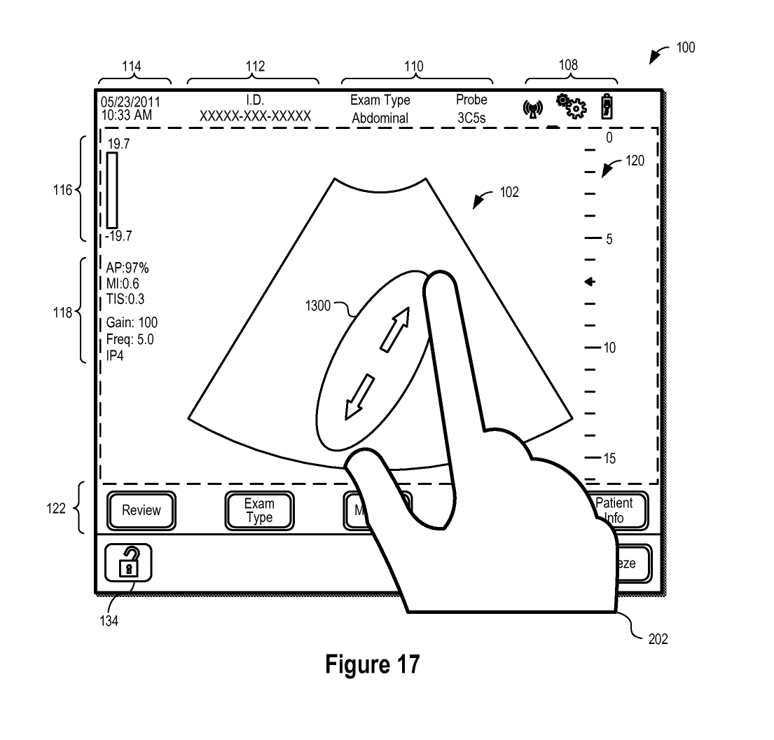

FIG. 17 illustrates another exemplary interface 100 for an ultrasound imaging system including a user-defined region of interest 1700 consistent with embodiments disclosed herein. Certain elements of the exemplary interface 100 may be similar to those illustrated and described in reference to FIGS. 1-16, and, accordingly, similar elements may be denoted with like numerals. In certain circumstances, a user 202 may wish to define a region of interest 1700 having a different shape than the region of interest 1300 illustrated previously (i.e., a non-parallelogram). For example, as illustrated in FIG. 17, a user 202 may wish to define a region of interest 1700 having a circular and/or oval shape.

To define a circular and/or oval region of interest 1300, the user 202 may interact directly with images displayed in the primary imaging area 102 by touching the interface 100 at a plurality of contact points within the primary imaging area 102. For example, as illustrated the user 202 may touch the interface 100 at two contact points. The user 202 may then define a circular and/or oval region of interest 1700 by utilizing a “spread” gesture on the interface 100 (e.g., by drawing two or more fingers apart while contacting the primary imaging area 102). The circular and/or oval region of interest 1700 may be displayed on the primary imaging area 102 centered between the two contact points. Any other suitable number of contact points, region of interest shapes, and/or gestures may also be utilized to define a region of interest 1700. The circular and/or oval region of interest 1700 may be resized and/or scaled using any other suitable gesture, as discussed in more detail above.

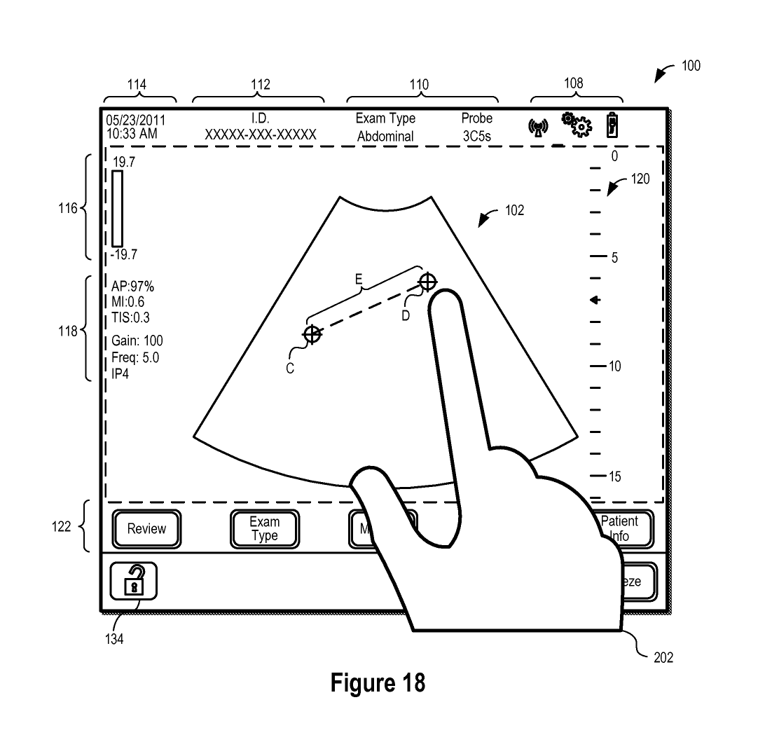

FIG. 18 illustrates another exemplary interface 100 for an ultrasound imaging system including a measurement system consistent with embodiments disclosed herein. Certain elements of the exemplary interface 100 may be similar to those illustrated and described in reference to FIGS. 1-17, and, accordingly, similar elements may be denoted with like numerals. As discussed above, a user 202 may utilize measurement functions accessed via a measure menu button that may allow the user 202 to measure certain portions of images displayed in the primary imaging area 102.

In certain embodiments, a user 202 may measure images displayed in the primary imaging area 102 by defining one or more measurement marker points within the displayed images. For example, as illustrated, a user 202 may define a first measurement marker point “C” within the primary imaging area 102. In certain embodiments, the first measurement marker point may be defined by positioning the measurement marker point “C” in a particular location in the primary imaging area 102 by touching the primary imaging area 102 at the particular location. The user 202 may place the measurement marker point “C” by using an appropriate gesture (e.g., a double tap at the location) on the primary imaging area 102. The user 202 may then define a second measurement marker point “D” within the primary imaging area 102 by positioning the measurement marker point “D” in a particular location in the primary imaging area 102 by touching the primary imaging area 102 at the particular location. The user 202 may place the measurement marker point “D” by using an appropriate gesture on the primary imaging area 102. The interface 100 may then display a measurement “E” indicating the relative distance between the measurement marker point “C” and measurement marker point.

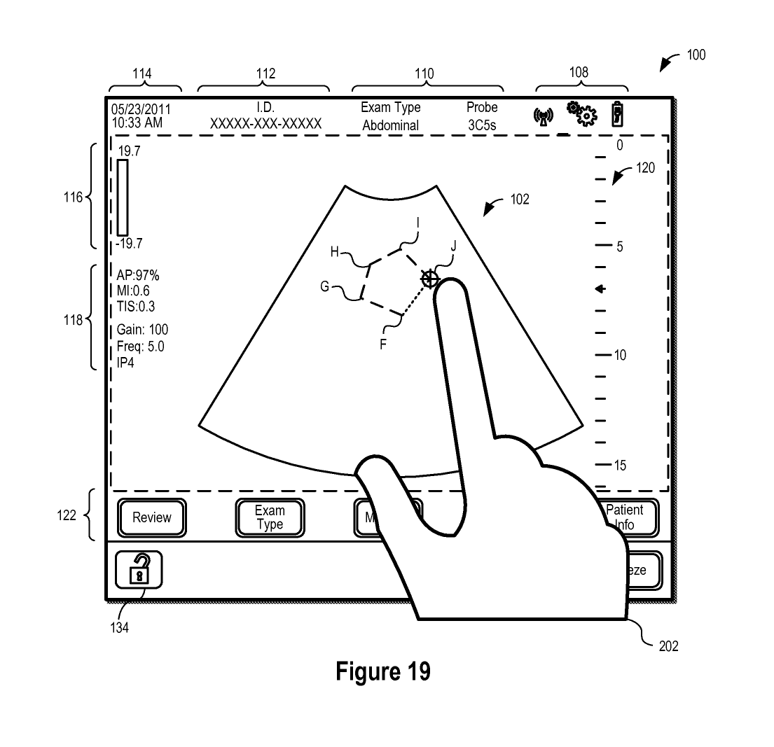

FIG. 19 illustrates another exemplary interface 100 for an ultrasound imaging system including multi-segment tracing consistent with embodiments disclosed herein. Certain elements of the exemplary interface 100 may be similar to those illustrated and described in reference to FIGS. 1-18, and, accordingly, similar elements may be denoted with like numerals. As discussed above, a user 202 may utilize tracing functions to perform multi-segment measurements of an image displayed in the primary imaging area 102. In certain embodiments, a multi-segment trace may be performed by placing one or more measurement marker points (e.g., measurement marker points “F”, “G”, “H”, “I”, and “J”) at particular locations in the primary imaging area 102 by interfacing with the primary imaging area 102 using suitable types of touch inputs such as those discussed previously. A tracing path may be defined having vertices corresponding to the measurement marker points. In certain embodiments, the interface 100 may be configured to automatically finalize a final segment of a tracing path by creating a segment between the first placed measurement marker point (e.g., point “F”) and a last placed measurement marker point (e.g., point “J”).

In some embodiments, the multi-segment trace path may be used for measurement purposes. For example, a measurement length of the multi-segment trace path may be displayed in the interface 100. In further embodiments, the multi-segment trace path may be utilized in zooming operations, in annotation operations, and/or the like.

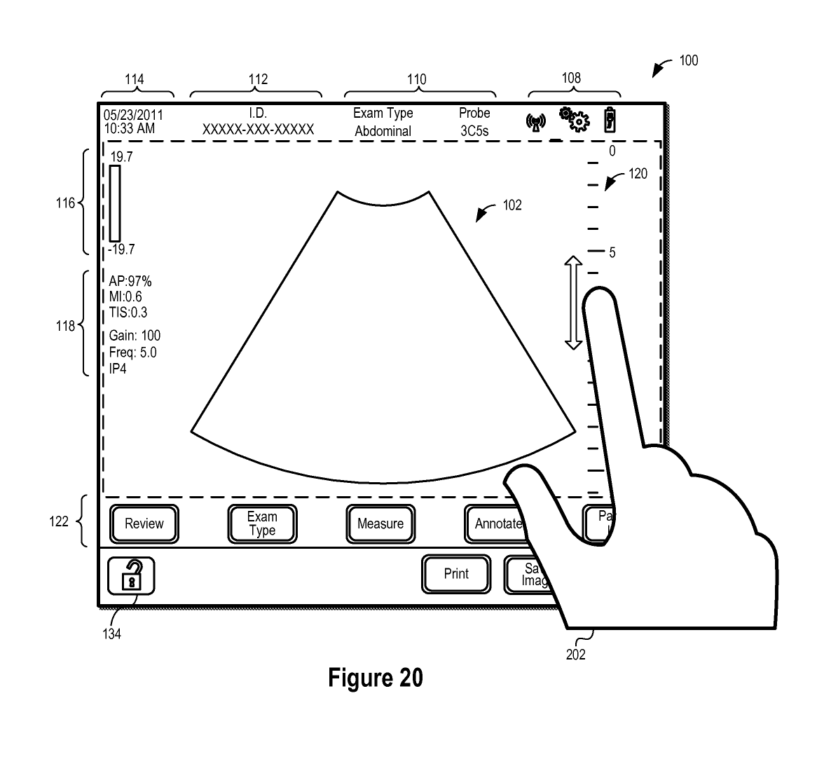

FIG. 20 illustrates an exemplary interface 100 for an ultrasound imaging system including scaling consistent with embodiments disclosed herein. Certain elements of the exemplary interface 100 may be similar to those illustrated and described in reference to FIGS. 1-19, and, accordingly, similar elements may be denoted with like numerals. In embodiments where the interface 100 is used to display representations of a 3-dimensional image (e.g., a 3-dimensional ultrasound image), the primary imaging area scale information 120 may be used to interpret and/or determine a relative depth of view within the 3-dimensional image. In certain embodiments, a user 202 may adjust the depth of view by touching the primary imaging area scale information 120 on the interface 100 and selecting an appropriate depth of view from the primary imaging area scale information 120. In the illustrated embodiments, depth of view may be adjusted by dynamically sliding a contact point with the interface 100 in an up and/or down direction along the primary imaging area scale information 120.

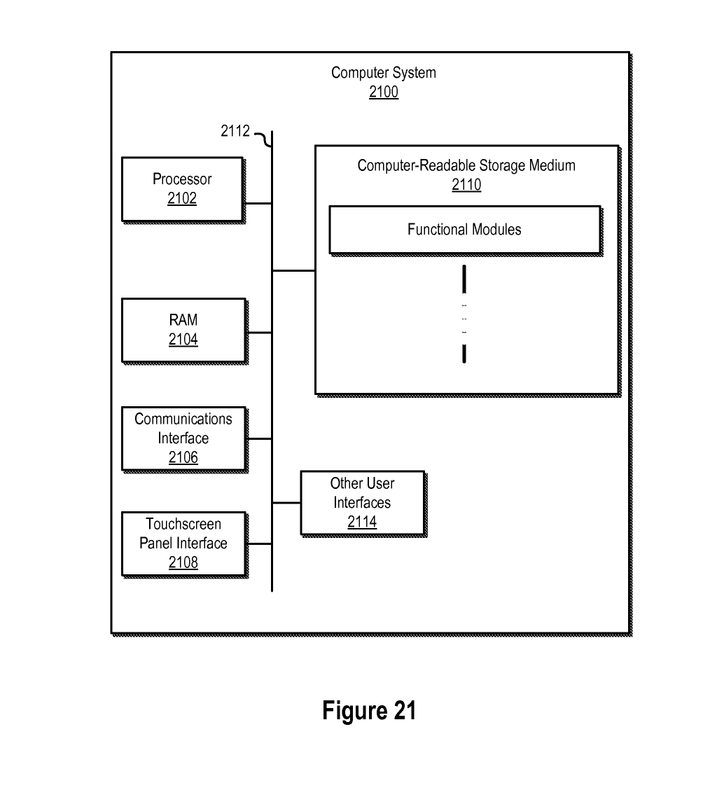

FIG. 21 illustrates a block diagram of a system 2100 for implementing certain embodiments disclosed herein. In certain embodiments, the system 2100 may be a discrete computing system incorporating a touch screen panel interface 2108 (e.g., an iPad or other suitable tablet computing device) implementing the interface 100 described above in reference to FIGS. 1-9 and configured to operate with other components of an ultrasound imaging system. In further embodiments, components of the system 2100 may be integrated as part of an ultrasound imaging system.

The system 2100 may include a processor 2102, a random access memory (“RAM”) 2104, a communications interface 2106, a touch screen panel interface 2108, other user interfaces 2114, and/or a non-transitory computer-readable storage medium 2110. The processor 2102, RAM 2104, communications interface 2106, touchscreen panel interface 2108, other user interfaces 2114, and computer-readable storage medium 2110 may be communicatively coupled to each other via a common data bus 2112. In some embodiments, the various components of the computer system 2100 may be implemented using hardware, software, firmware, and/or any combination thereof.

The touchscreen panel interface 2108 may be used to display an interactive interface to a user such as, for example, the interface 100 described in reference to and illustrated in FIGS. 1-20. The touchscreen panel interface 2108 may be integrated in the computer system 2100 or, alternatively, may be a discrete touchscreen panel interface 2108 from a touchscreen laptop or tablet computer communicatively coupled with the computer system 2100. The communications interface 2106 may be any interface capable of communicating with other computer systems and/or other equipment (e.g., remote network equipment) communicatively coupled to computer system 2100. The other user interfaces 2114 may include any other user interface a user 202 may utilize to interact with the computer system 2100 including, for example, a keyboard, a mouse pointer, a joystick, and the like.

The processor 2102 may include one or more general purpose processors, application specific processors, microcontrollers, digital signal processors, FPGAs, or any other customizable or programmable processing device. The processor 2102 may be configured to execute computer-readable instructions stored on the non-transitory computer-readable storage medium 2110. In some embodiments, the computer-readable instructions may be computer executable functional modules. For example, the computer-readable instructions may include one or more functional modules configured to implement all or part of the functionality of the systems, methods, and interfaces described above in reference to FIGS. 1-20.

Some of the infrastructure that can be used with embodiments disclosed herein is already available, such as general-purpose computers, ultrasound imaging systems, touch screen panels, computer programming tools and techniques, digital storage media, and communications networks. A computing device may include a processor such as a microprocessor, microcontroller, logic circuitry, or the like. The processor may include a special purpose processing device such as an ASIC, PAL, PLA, PLD, FPGA, or other customized or programmable device. The computing device may also include a computer-readable storage device such as non-volatile memory, static RAM, dynamic RAM, ROM, CD-ROM, disk, tape, magnetic, optical, flash memory, or other computer-readable storage medium.

Various aspects of certain embodiments may be implemented using hardware, software, firmware, or a combination thereof. As used herein, a software module or component may include any type of computer instruction or computer executable code located within or on a non-transitory computer-readable storage medium. A software module may, for instance, comprise one or more physical or logical blocks of computer instructions, which may be organized as a routine, program, object, component, data structure, etc., that performs one or more tasks or implements particular abstract data types.

In certain embodiments, a particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media. Some embodiments may be practiced in a distributed computing environment where tasks are performed by a remote processing device linked through a communications network.

The systems and methods disclosed herein are not inherently related to any particular computer or other apparatus and may be implemented by a suitable combination of hardware, software, and/or firmware. Software implementations may include one or more computer programs comprising executable code/instructions that, when executed by a processor, may cause the processor to perform a method defined at least in part by the executable instructions. The computer program can be written in any form of programming language, including compiled or interpreted languages, and can be deployed in any form, including as a standalone program or as a module, component, subroutine, or other unit suitable for use in a computing environment. Further, a computer program can be deployed to be executed on one computer or on multiple computers at one site or distributed across multiple sites and interconnected by a communication network. Software embodiments may be implemented as a computer program product that comprises a non-transitory storage medium configured to store computer programs and instructions that, when executed by a processor, are configured to cause the processor to perform a method according to the instructions. In certain embodiments, the non-transitory storage medium may take any form capable of storing processor-readable instructions on a non-transitory storage medium. A non-transitory storage medium may be embodied by a compact disk, digital-video disk, a magnetic tape, a Bernoulli drive, a magnetic disk, a punch card, flash memory, integrated circuits, or any other non-transitory digital processing apparatus memory device.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive, and the invention is not to be limited to the details given herein, but may be modified within the scope and equivalents of the appended claims.

The foregoing specification has been described with reference to various embodiments. However, one of ordinary skill in the art will appreciate that various modifications and changes can be made without departing from the scope of the present disclosure. For example, various operational steps, as well as components for carrying out operational steps, may be implemented in alternate ways depending upon the particular application or in consideration of any number of cost functions associated with the operation of the system. Accordingly, any one or more of the steps may be deleted, modified, or combined with other steps. Further, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced, are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

Those having skill in the art will appreciate that many changes may be made to the details of the above-described embodiments without departing from the underlying principles of the invention. The scope of the present invention should, therefore, be determined only by the following claims.

What is claimed is:

1. A medical imaging system comprising a touch screen display, a processor, and a non-transitory computer-readable medium, the non-transitory computer-readable medium storing instructions that, when executed by the processor, cause the processor to:
receive an input from a user based on a position of a contact point of the user with the touch screen display, the contact point being located within a primary imaging area of the touch screen display displaying one or more medical images captured by the medical imaging system; and
display, in response to the input, a cursor on the touch screen display within the primary imaging area at a particular position relative to the position of the contact point, the cursor being at a particular distance and orientation from the position of the contact point.

2. The medical imaging system of claim 1, wherein the medical imaging system comprises an ultrasound imaging system.

3. The medical imaging system of claim 1, wherein the particular position is an offset position relative to the position of the contact point.

4. The medical imaging system of claim 1, wherein changes in the position of the contact point are translated into respective changes in a position of the cursor.

5. The medical imaging system of claim 1, wherein the cursor comprises an annotation.

6. The medical imaging system of claim 1, wherein the cursor comprises a measurement marker point.

7. The medical imaging system of claim 1, wherein a line is displayed between the position of the contact point and the cursor.

8. The medical imaging system of claim 1, wherein the particular position is a position wherein a user does not obscure the cursor when contacting the touch screen display at the position of the contact point.

9. A medical imaging system comprising:
an imaging system configured to capture one or more medical images; and
a touch screen display communicatively coupled with the imaging system, the touch screen display being configured to:
receive an input from a user based on a position of a contact point of the user with the touch screen display, the contact point being located within a primary imaging area of the touch screen display displaying the one or more medical images; and
display, in response to the input, a cursor within the primary imaging area at a particular position relative to the position of the contact point, the being at a particular distance and orientation from the position of the contact point.

10. The medical imaging system of claim 9, wherein the imaging system comprises an ultrasound imaging system.

11. The medical imaging system of claim 9, wherein the particular position is an offset position relative to the position of the contact point.

12. The medical imaging system of claim 9, wherein changes in the position of the contact point are translated into respective changes in a position of the cursor.

13. The medical imaging system of claim 9, wherein the cursor comprises an annotation.

14. The medical imaging system of claim 9, wherein the cursor comprises a measurement marker point.

15. The medical imaging system of claim 9, wherein a line is displayed between the position of the contact point and the cursor.

16. The medical imaging system of claim 9, wherein the particular position is a position wherein the user does not obscure the cursor when contacting the touch screen display at the position of the contact point.
